SEQUENCE LISTING (28 sequences)

SEQ ID NO: 1 - protein, 116 aa, Bacillus subtilis
MIVKRGDVYFADLSPVVGSEQGGVRPVLVIQNDIGNRFSPTAIVAAITAQIQKAKLPTHVEIDAKRYGFERDSVILLEQIRTIDKQRLTDKITHLDDEMMDKVDEALQISLALIDF

SEQ ID NO: 2 - protein, 120 aa, Staphylococcus aureus
MIRRGDVYLADLSPVQGSEQGGVRPVVIIQNDTGNKYSPTVIVAAITGRINKAKIPTHVEIEKKKYKLDKDSVILLEQIRTLDKKRLKEKLTYLSDDKMKEVDNALMISLGLNAVAHGKN

SEQ ID NO: 3 - protein, 111 aa, Escherichia coli
MVSRYVPDMGDLIWVDFDPTKGSEQAGHRPAVVLSPFMYNNKTGMCLCVPCTTQSKGYPFEVVLSGQERDGVALADQVKSIAWRARGATKKGTVAPEELQLIKAKINVLIG

SEQ ID NO: 4 - protein, 110 aa, Escherichia coli
MERGEIWLVSLDPTAGHEQQGTRPVLIVTPAAFNRVTRLPVVVPVTSGGNFARTAGFAVSLDGVGIRTTGVVRCDQPRTIDMKARGGKRLERVPETIMNEVLGRLSTILT

SEQ ID NO: 5 - protein, 116 aa, Escherichia coli
MVKKSEFERGDIVLVGFDPASGHEQQGAGRPALVLSVQAFNQLGMTLVAPITQGGNFARYAGFSVPLHCEEGDVHGVVLVNQVRMMDLHARLAKRIGLAADEVVEEALLRLQAVVE

SEQ ID NO: 6 - protein, 118 aa, Mycobacterium tuberculosis
MMRRGEIWQVDLDPARGSEANNQRPAVVVSNDRANATATRLGRGVITVVPVTSNIAKVYPFQVLLSATTTGLQVDCKAQAEQIRSIATERLLRPIGRVSAAELAQLDEALKLHLDLWS

SEQ ID NO: 7 - protein, 114 aa, Mycobacterium tuberculosis
MVISRAEIYWADLGPPSGSQPAKRRPVLVIQSDPYNASRLATVIAAVITSNTALAAMPGNVFLPATTTRLPRDSVVNVTAIVTLNKTDLTDRVGEVPASLMHEVDRGLRRVLDL

SEQ ID NO: 8 - protein, 103 aa, Mycobacterium tuberculosis
MRPIHIAQLDKARPVLILTREVVRPHLTNVTVAPITTTVRGLATEVPVDAVNGLNQPSVVSCDNTQTIPVCDLGRQIGYLLASQEPALAEAIGNAFDLDWVVA

SEQ ID NO: 9 - protein, 105 aa, Mycobacterium tuberculosis
MMAPLRGQVYRCDLGYGAKPWLIVSNNARNRHTADVVAVRLTTTRRTIPTWVAMGPSDPLTGYVNADNIETLGKDELGDYLGEVTPATMNKINTALATALGLPWP

SEQ ID NO: 10 - protein, 122 aa, Myxococcus xanthus
MPPERINRGDVFWVEPDDSRGPVPSYSHPHVVVQDDVFNHSRITTVVVCALTSNLHRASEPGNVLLEVGEGNLPKQSVVVVSQVSSVDKARLGERIGALSDARVEQILAGLRFQQVSFFARP

SEQ ID NO: 11 - protein, 124 aa, Haloquadra walsbyi
MVTPRCRYVQVRRGDIVIVDLSPTKGSEQQGTNRPCVVIQNDVGNRNSPTTIIAPFTKQYNPDNTYPFEVEVLASNTSLNQDSVADLSQIRVVDINKGVKTNIGSVPSARMAKIDTAIKTSLGL

SEQ ID NO: 12 - DNA, 369 bp, Bacillus subtilis
atgattgtgaaacgtggcgatgtgtattttgcggatctgagcccggtggtgggcagcgagcagggcggcgtgcgtccggtgctggtgattcagaacgatattggcaaccgttttagcccgaccgcgattgtggcggcgattaccgcgcagattcagaaagcgaaactgccgacccatgtggaaattgatgcgaaacgttatggctttgaacgtgatagcgtgattctgctggagcagattcgtaccattgataagcagcgtctgaccgataaaattacccatctggatgatgaaatgatggataaagtggatgaagcgctgcagattagcctggcgctgattgattttcatcatcatcatcatcattaa

SEQ ID NO: 13 - protein, 122 aa, Bacillus subtilis
MIVKRGDVYFADLSPVVGSEQGGVRPVLVIQNDIGNRFSPTAIVAAITAQIQKAKLPTHVEIDAKRYGFERDSVILLEQIRTIDKQRLTDKITHLDDEMMDKVDEALQISLALIDFHHHHHH

SEQ ID NO: 14 - RNA, 15 nt, Bacillus subtilis
gcuccuacaugucag

SEQ ID NO: 15 - RNA, 15 nt, Bacillus subtilis
gguuuuacauaaacg

SEQ ID NO: 16 - RNA, 15 nt, Bacillus subtilis
gguuuuacauaaacg

SEQ ID NO: 17 - RNA, 15 nt, Bacillus subtilis
ugacuuacaucgaag

SEQ ID NO: 18 - RNA, 15 nt, Bacillus subtilis
cguuuuacaucaaga

SEQ ID NO: 19 - RNA, 15 nt, Bacillus subtilis
gucgcuacauagcgu

SEQ ID NO: 20 - RNA, 13 nt, Artificial
  Synthetic
cucnuacauauca

SEQ ID NO: 21 - RNA, 13 nt, Artificial
  Synthetic
cucuuacauauca

SEQ ID NO: 22 - RNA, 13 nt, Artificial
  Synthetic
cucauacauauca

SEQ ID NO: 23 - RNA, 13 nt, Artificial
  Synthetic
cucguacauauca

SEQ ID NO: 24 - RNA, 13 nt, Artificial
  Synthetic
cuccuacauauca

SEQ ID NO: 25 - RNA, 13 nt, Artificial
  Synthetic
cucuuacaunuca

SEQ ID NO: 26 - RNA, 13 nt, Artificial
  Synthetic
cucuuacauauca

SEQ ID NO: 27 - RNA, 13 nt, Artificial
  Synthetic
cucuuacauguca

SEQ ID NO: 28 - RNA, 13 nt, Artificial
  Synthetic
cucuuacaucuca

(19) United States
(12) Patent Application Publication
(10) Pub. No.: US 2014/0319415 A1
(43) Pub. Date: Oct. 30, 2014

(54) METHODS OF INCORPORATING AMINO ACID ANALOGUES INTO A PROTEIN

(71) Applicant: Rutgers, The State University of New Jersey, New Brunswick, NJ (US)

(72) Inventors: Masayori Inouye, Highland Park, NJ (US); Yojiro Ishida, Highland Park, NJ (US); Lili Mao, St. Anthony, MN (US); Yoshihiro Yamaguchi, Somerset, NJ (US); Jung Ho Park, Seoul (KR)

(73) Assignee: Rutgers, The State University of New Jersey, New Brunswick, NJ (US)

(21) Appl. No.: 14/212,732

(22) Filed: Mar. 14, 2014

Related U.S. Application Data
(60) Provisional application No. 61/783,497, filed on Mar. 14, 2013.

(51) Int. Cl. C12P 21/00

(52) U.S. Cl. CPC C12P 21/00
USPC 252/182.12; 435/69.1; 530/350

(57) ABSTRACT

The present invention provides a novel use of a SPP system for replacing natural amino acid residues with non-naturally occurring amino acids in a protein or peptide, using cell based expression system.

### CROSS-REFERENCE TO RELATED APPLICATION

This application claims the benefit of priority to U.S. Provisional Patent Application No. 61/783,497 filed on Mar. 14, 2013, the disclosure of which is incorporated herein by reference.

### GOVERNMENT INTERESTS

This invention was made with government support under grant number R01 GM081567 from the National Institutes of Health. The United States government has certain rights to this invention.

### FIELD OF THE INVENTION

The present invention relates to a novel method of producing altered peptides and proteins by substituting amino acid residues with specific non-natural amino acid residues.

### BACKGROUND OF THE INVENTION

There are a large number of non-natural amino acid analogues. It is quite intriguing to replace all residues of a specific amino acid in a protein with its analogues, as it may create novel functional proteins with altered structures. However this is highly challenging as most amino acid analogues are highly toxic to the cells. To circumvent this problem, chemically modified aminoacylated tRNAs have been used in a cell-free system. Alternatively, an orthogonal aminoacyl tRNA synthetases/tRNA pair from other species was incorporated into bacteria or eukaryotes. One such highly toxic analogue is L-canavanine [L-2-amino-4-(guanidinooxy)butyric acid] (Can), an Arg analogue (FIG. 1A, B), which is found as an insecticide in certain leguminous plants such as jack bean. Its incorporation into cellular proteins leads to the production of functionally aberrant proteins, leading to failure of various cellular functions, causing eventual cell death. In a previous attempt, 18 out of 200 Arg residues in vitellogenin, an egg yolk protein, in locusts were replaced with Can, resulting in the production of an aberrantly structured protein, while one-third of three Arg residues in diptericin A, an antibacterial protein from the fly, Phormia terranovoe, was replaced with Can resulting in loss of the antibacterial activity. In another attempt, 21% of Arg residues in the lysozyme molecule from the tobacco hornworm, Manduca sexta, were replaced with Can, resulting in loss of 49.5% of the catalytic activity. None of these attempts, however, could achieve the complete replacement of all Arg residues in a protein with Can. RNA-mediated mRNA interference with the use of antisense RNA, miRNA and siRNA has been well documented, including their important roles in gene regulation from bacteria to human cells, and a possible use for the treatment of human diseases. More recently, it has been shown that mRNA interference is also mediated by proteins using sequence-specific endoribonucleases, called mRNA interferases. The first such enzyme reported was MazF-ec from Escherichia coli consisting of 111 residues, which cleaves RNA specifically at ACA sequences. The X-ray structure of its complex with the cognate antitoxin, MazE, has been determined, consisting of one MazE dimer with two MazF dimmers. Since then, a number of MazF homologues have been discovered from bacteria and archaea (FIG. 1C). Most recently, a seven-base specific MazF homologue from a super halophilic archaeon from a hypersaline pool on the Sinai Peninsula (MazF-hw) was found to cleave RNA at UUACUCA, which can be used for regulating specific gene expression in E. coli. There is a need to develop this technology.

### SUMMARY OF THE INVENTION

The present invention relates to methods of producing a target protein containing an amino acid analogue or amino acid analogues within the primary amino acid sequence of the protein which replace a normal amino acid within a target protein. The host cell expression system may be a eukaryotic or prokaryotic cell-based expression system.

The present invention further relates to methods of producing a protein containing an amino acid analogue(s) within the primary amino acid sequence of the protein via utilization of a prokaryotic cell-based expression system, including but in no way limited to an Escherichia coli cell-based expression system, wherein such a cell-based expression system (i) contains a trans-acting factor which substantially inhibits or degrades host cell RNA transcripts in conjunction with an expressed mRNA encoding the target protein which is not susceptible to such degradation; (ii) substantially prevents incorporation of the respective amino acid analogue into host cellular or non-target proteins, and (iii) substantially prevents de novo biosynthesis of the particular proteinogenic amino acid targeted for replacement or substitution. The term “trans-acting factor” as used herein refers to “any protein or any other component” which acts to substantially inhibit[s] or degrade[s] mRNA.

To this end, the present invention relates to a method of producing a target protein containing the targeted replacement of a natural amino acid with an amino acid analogue within a eukaryotic or prokaryotic host cell-based expression system, including but in no way limited to an Escherichia coli cell-based expression system, which comprises (i) introducing into a host cell an expression vector which expresses a protein which substantially cleaves host mRNA; (ii) introducing into the host cell an expression vector encoding a mRNA transcript which expresses the target protein of interest, the mRNA encoding the target protein being resistant to cleavage by the protein of (i) which selectively degrades host cell mRNA; (iii) inducing expression of the protein of (i) within the host cell; (iv) removing from the host cell culture environment the natural amino acid targeted for replacement; (v) adding to the host cell culture environment the amino acid analogue; (vi) inducing expression of the target protein of step (ii) within the host cell; and, (vii) purifying the expressed target protein of (vi) away from the host cell components, wherein (a) incorporation of the amino acid analog(s) into host cellular proteins other than the target protein is substantially prevented within the host cell, and (b) de novo biosynthesis of the replaced natural amino acid is substantially inhibited within the host cell.

A particular embodiment of the present invention relates to use of a single protein production system (SPP), as disclosed herein and shown schematically at FIG. 2B, and as further disclosed in Vaiphei et al. (2010) Appl. Environ. Microbiol. 76: 6063-6068 and Suzuki et al. (2006) J. Biol. Chem. 281: 37599-37565, both references which are incorporated by reference herein in their entirety. Another particular embodiment of this portion of the present invention utilizing SPP-based expression includes, but is in no way limited to, the use of a host cell expression of MazF-ec to promote a first component of substantially inhibiting or removing host RNA transcripts.

Another embodiment of the present invention relates to use of a prokaryotic cell-based expression system as disclosed herein whereby a target protein as disclosed in FIG. 1C is generated which substitutes or replaces each arginine (Arg) residue with canavanine (Can), including but in no way limited to an Escherichia coli cell-based expression system, wherein such a cell-based expression system (i) contains a trans-acting factor which substantially inhibits or degrades host cell RNA transcripts in conjunction with an expressed mRNA encoding the target protein which is not susceptible to such degradation; (ii) substantially prevents incorporation of the respective amino acid analogue into host cellular or non-target proteins, and (iii) substantially prevents de novo biosynthesis of the particular proteinogenic amino acid targeted for replacement or substitution by an amino analogue.

A further embodiment relates to the methods disclosed herein for use in a prokaryotic cell-based expression system, including but not limited to an Escherichia coli cell-based expression system whereby a target protein selected from the group consisting of MazF-bs and MazF-hw, as disclosed in FIG. 1C, are generated which substitutes or replaces each arginine (Arg) residue with canavanine (Can) to produce MazF-bs (can) and MazF-hw (can).

Another embodiment relates to the use of an an Escherichia coli cell-based expression system as shown schematically in FIG. 2B and as exemplified herein, to produce MazF-bs (can), a protein where all seven arginine residues (as disclosed in FIG. 1C and FIG. 2A) are replaced with a canavanine residue.

The present invention further relates to isolated proteins which comprise one or more substituted or replaced amino acid(s) within the primary amino acid sequence of the isolated protein with an amino acid analogue(s) as compared to the respective amino acid sequences disclosed in FIG. 1A, namely, MazF-bs, MazF-sa, MazF-ec, PemK-ec, ChpBK-ec, MazF-mt1, MazF-mt3, MazF-mt6, MazF-mt7, MazF-mx, and MazF-hw.

The present invention further relates to isolated proteins which comprise an amino acid sequence whereby each arginine residue has been substituted with a canavanine residue compared to the respective amino acid sequences disclosed in FIG. 1A, namely, MazF-bs, MazF-sa, MazF-ec, PemK-ec, ChpBK-ec, MazF-mt1, MazF-mt3, MazF-mt6, MazF-mt7, MazF-mx, and MazF-hw, resulting in MazF-bs (can), MazF-sa (can), MazF-ec (can), PemK-ec (can), ChpBK-ec (can), MazF-mt1 (can), MazF-mt3 (can), MazF-mt6 (can), MazF-mt7 (can), MazF-mx, and MazF-hw (can), respectively.

A particular embodiment of this portion of the present invention relates to isolated proteins selected from the group consisting of MazF-bs (can) and MazF-hw (can), respectively.

Another particular embodiment of this portion of the present invention relates to the isolated protein MazF-bs (can), which substitutes canavanine for each of the seven arginine residues of MazF-bs, as disclosed in FIG. 1A.

Thus, replacement of a specific amino acid residue in a protein with non-natural analogues is highly challenging because of their cellular toxicity. Exemplified herein is the use of a cell-based expression system to replace all arginine (Arg) residues in a protein with canavanine (Can), a toxic Arg analogue. All Arg residues in the five-base specific (UACAU) mRNA interferase from Bacillus subtilis [MazF-bs(arg)] were replaced with Can by using the Single-Protein Production system in Escherichia coli. The resulting MazF-bs(can) gained a six-base recognition sequence, UACAUA, for RNA cleavage instead of the five-base sequence, UACAU, for MazF-bs(arg). Mass spectrometry analysis confirmed that all Arg residues were replaced with Can. The present system offers a novel approach to create new functional proteins by replacing a specific amino acid in a protein with its analogues.

The present invention also relates to a method of using a SPP system for replacing at least one arginine with at least one canavanine in a peptide or protein, comprising:
a. transforming BL21(DE3) (ΔargHΔtrpCΔhisB) cells with a vector containing an ACA-less gene for a Protein Y together with pACYCmazF(ΔH);
b. inducing protein expression of MazF(ΔH);
c. incorporating canavanine into Protein Y;
d. purifying Protein Y(arg) and Protein Y(can); and
e. further purifying Protein Y(can).

As noted herein, the present invention relates to a composition comprising MazF-bs(can).

As noted herein, the present invention relates to a composition comprising Protein Y(can).

The present invention additionally relates to a method of using a SPP system for replacing at least one arginine with at least one canavanine in a peptide or protein, comprising:
a. transforming BL21(DE3) (ΔargHΔtrpCΔhisB) cells with a vector containing an ACA-less gene for a protein (Protein Y) together with pACYCmazF(ΔH);
b. inducing expression of MazF(ΔH) in a medium containing Arg, Trp and His;
c. incorporating canavanine into Protein Y by replacing Arg with canavanine in the medium;
d. purifying Protein Y(can); and
e. purifying Protein Y(arg) from the medium containing Arg.

The preferred methods and materials are described below in examples which are meant to illustrate, not limit, the invention. Skilled artisans will recognize methods and materials that are similar or equivalent to those described herein, and that can be used in the practice or testing of the present invention.

### BRIEF DESCRIPTION OF THE FIGURES

FIG. 1 illustrates the structures of L-canavanine and L-arginine and the tertiary structure and multiple alignment of MazF-bs. The structures of L-canavanine (A) and L-arginine (B). C, alignments of MazF-bs with other MazF homologues from Staphylococcus aureus (MazF-sa) and E. coli (MazF-ec), PemK from E. coli (PemK-ec), ChpBK from E. coli (ChpBK-ec), MazF-mt1 to -mt7 from Mycobacterium tuberculosis, MazF-mx from Myxococcus xanthus, and MazF-hw from Haloquadra walsbyi. White and black boxes indicate β-sheets and α-helices on the basis of the secondary structure of MazF-bs, respectively. ClustalW2 was used for multiple sequence alignments. All Arg residues in MazF-bs are indicated by black shading, and homologous residues to Arg by gray shading. D, The crystal structure of MazF-bs dimer (PDB ID: 1NE8) is imaged in PyMOL. Dark and light shading indicate two individual monomers and Arg residues are designated by “RXX” wherein XX indicates position of the Arg residue.

FIG. 2 illustrates schematic procedures for the production of MazF-bs(arg) and MazF-bs(can). FIG. 2A illustrates the DNA sequence of the mazF-bs gene. The gene is designed to be ACA-less and codon-optimized for E. coli. The amino acid sequence of MazF-bs is shown under the DNA sequence. FIG. 2B illustrates the dual inducible Single-Protein-Production (SPP) system. The BL21(DE3) (ΔargHΔtrpCΔhisB) cells were transformed with pColdIIImazF-bs together with pACYCmazF(ΔH) and grown in a 1-liter culture of M9-glucose medium in the presence of Arg (20 μg/ml), His (20 μg/ml), and Trp (20 μg/ml) at 37° C. When the A600 value reached 0.5, the culture was chilled in an ice-water bath for 5 min and incubated at 15° C. for 1 hr to acclimate the cells to cold shock conditions. Cells were harvested and washed twice with M9 medium. The cells were resuspended in 50 ml of M9-glucose medium containing Arg (20 μg/ml) and Trp (20 μg/ml) but without His. Isopropyl β-D-1-thiogalactopyranoside (IPTG; 0.5 mM) was added to induce the only expression of MazF(ΔH) followed by an additional 2 hr incubation at 15° C. Cells were harvested and washed twice with M9 medium. The cells were resuspended in 50 ml of M9-glucose medium containing His (20 μg/ml), and Trp (20 μg/ml), Can (100 μg/ml) and IPTG (0.5 mM) to incorporate Can into MazF-bs. The cell culture was incubated at 15° C. for additional 24 hr to induce MazF-bs(can). When Arg (20 μg/ml) was added instead of Can, MazF-bs(arg) was produced.

FIG. 3 illustrates the analysis of the secondary structures of MazF-bs(can) and MazF(can). FIG. 3A illustrates purified MazF bs(arg) (lane 1) and MazF-bs(can) (lane 2) as descrete bands in 17% SDS-PAGE stained with Coomassie Blue. FIG. 3B illustrates total mass measurement by MALDI-TOF (Applied Biosystem) of MazF-bs(arg) and MazF-bs(can), respectively. FIG. 3C illustrates CD spectra of the secondary structures of MazF-bs(arg) and MazF-bs(can). FIG. 3D illustrates thermal stabilities of MazF-bs(arg) and MazF-bs(can). The open and black circles represent MazF-bs(can) and MazF-bs(arg), respectively.

FIG. 4 illustrates the acquirement of a higher RNA cleavage specificity in MazF-bs(can). FIG. 4A illustrates the analysis of cleavage sites in MS2 phage RNA by MazF-bs(arg) or MazF-bs(can). Lane C represents a control reaction in which no protein was added; MS2 RNA was incubated with MazF-bs(arg) or MazF-bs(can) for 1, 5, 10, 30 min [Lanes 1-4; MazF-bs(arg), 5-8; MazF-bs(can)]. A black arrow indicates a full length (3.5 kb) of MS2 phage RNA. FIG. 4B-F illustrates the analysis of MazF-bs(can) cleavage sites in MS2 phage RNA by in vitro primer extension. Each panel represents different UACAU sites in MS2 RNA. Lane 1; MS2 RNA was incubated with purified CspA. Lanes 2 and 3; MS2 RNA was incubated with MazF-bs(can) and MazF-bs(arg) in the presence of CspA, an RNA chaperone, respectively. G, A, U, and C with an upper black bar indicate the sequence ladder for each reaction primer. Ribonucleotide sequences in each panel (B-F) indicate the cleavage sequences for MazF-bs(can) and MazF-bs(arg). The ÛACAU sequences with an under bar indicate the cleavage sites by MazF-bs(arg), and one extra ribonucleotide, A, at the 3′ end, adjacent to the cleavage site, which is required for the cleavage by MazF-bs(can).

FIG. 5 illustrates the identification of a change of RNA cleavage specificity in MazF-bs(can). FIG. 5A illustrates the 13-base ribonucleotides (CUCXUACAUAUCA) synthesized, where the 4th base (X) was U, A, G, or C, were incubated with MazF-bs(can) or MazF-bs(arg) (lanes 2-5, and 7-10, respectively). Lanes 1 and 6 represent control reactions in which no protein was added. Lanes 2 and 7 show an extra band corresponding to the product cleaved after the first C residue in addition to the cleavage product after the fifth U residue ĈUCUÛACAUAUCA (̂ indicates the cleavage sites), while no cleavage products were observed with three other ribonucleotides (CUCAUACAUAUCA, CUCGUACAUAUCA, CUCCUACAUAUCA) for both MazF-bs(can) and MazF-bs(arg) (bases which are replaced are shown in bold). FIG. 5B illustrates the 13-base ribonucleotides (CUCUUACAUYUCA) synthesized, where the Y position was A, U, G or C, were incubated with MazF-bs(can) or MazF-bs(arg) (lanes 2-5, and 7-10, respectively). Lanes 1 and 6 represent control reactions in which no protein was added. The lane 2 shows an extra cleavage product (cleaved after the first C residue) in addition to the product cleaved after the fifth U residue. Lanes 7, 8 and 10 also show an extra cleavage product corresponding to ĈUCUUACAUAUCA, ĈUCUUACAUGUCA, ĈUCUUACAUCUCA. These cleavages were not observed with MazF-bs(can).

### DETAILED DESCRIPTION OF INVENTION

The present invention relates to methods of producing a protein containing an amino acid analogue or amino acid analogues within the primary amino acid sequence of the protein via utilization of a eukaryotic or prokaryotic cell-based expression system. These methods relate to production of a protein containing one or more such non-natural or analogue amino acid(s) within the primary amino acid sequence of the protein via utilization of a cell-based expression system, wherein replacement of any such natural amino acid(s) with a non-natural amino acid(s) results in a ‘nonnatural’ protein with a biological activity or function which may differ from the wild type or template protein. The term ‘normal’ or ‘natural’ or ‘proteogenic’ amino acid as used herein refers to a least one of the twenty amino acids that make up the structural units of proteins, namely alanine (Ala), glycine (Gly), valine (Val), leucine (Leu), isoleucine (Ile), proline (Pro), phenylalanine (Phe), tyrosine (Tyr), tryptophan (Trp), serine (Ser), threonine (Thr), cysteine (Cys), methionine (Met), as asparagine (Asn), glutamine (Gln), lysine (Lys), arginine (Arg), histidine (His), aspartate (Asp), and glutamate (Glu). Amino acid analogues include but are not limited to Azetidine-2-carboxylic acid, 3,4-Dehydroproline, Perthiaproline, Canavanine, Ethionine, Norleucine, Selenomethionine, Aminohexanoic acid, Aminohexanoic acid, Telluromethionine, Homoallylglycine, Homopropargylglycine, 2-Butynylglycine, Azidohomolanine, Transcrotylglycine, Allyglycine, 7-Azatryptophan, 4-Fluorotryptophan, 5-Fluorotryptophan, 7-Fluorotryptophan, J3-(Thienopyrrolyl)alanines, J3-Selenolo(3,3-J3)pyrrolyl-alanine, Aminotryptophans, Trifluoroleucine and Norvaline. See for example, Hendrickson et al (2004) Annu Rev. Biochem. 73: 147-176, which is incorporated herein by reference.

As used herein, the term ‘amino acid analogue’ is interchangeable with the term ‘non-natural amino acid.’ It will be understood that the methods of the present invention may be applied to incorporate any known, specified amino acid analogue into the expressed protein whereby that amino acid analogue is recognized by a respective host cell aminoacyl-tRNA synthetase. To this end, the artisan, with the aid of this disclosure, is presented the opportunity to easily test any potential amino acid analogue of interest to determine whether the required compatibility exists between the non-natural amino acid analogue and the array of aminoacyl-tRNA synthetases available within the respective host cell environment.

It is well within the scope of this disclosure to utilize a host strain which encodes a mutated aminoacyl tRNA synthetase which is effectively modified to carry and attach to a non-natural amino acid as disclosed herein. It is known to the artisan that a mutant synthetase may then be genetically programmed to incorporate a non-natural amino acid into any desired position in any protein of interest.

One embodiment of the present invention relates to methods of producing a protein as disclosed herein which contains one or more amino acid analogue(s) within the primary amino acid sequence of the protein via utilization of a prokaryotic cell-based expression system. It is understood that the target protein may have one ore more naturally occurring amino acids, which would still result in a protein with a modified function as compared to wild-type.

Another embodiment of the present invention relates to methods of producing a protein containing an amino acid analogue(s) within the primary amino acid sequence of the protein via utilization of an Escherichia coli cell-based expression system.

One aspect of the present invention relates to a method of producing a protein containing an amino acid analogue(s) within the primary amino acid sequence of the protein via utilization of a cell-based expression system, wherein a trans-acting factor is utilized as a component to specifically and substantially inhibit or remove host cell RNA transcripts in order to prevent translation of host cell proteins containing amino acid analogue residues without having an adverse effect on the target protein containing said amino acid analogue. Simply presented as an example, but in no way a limitation, it is disclosed herein that one avenue of inhibiting translation of such host cell RNA transcripts early on during the cell culture process may be accomplished by expressing or presenting MazF-ec protein within the host cell in later combination with expression of a target protein which contains no MazF-ec-cleavable ‘ACA’ nucleotide sequence within the open reading frame of the respective target gene mRNA, whereby host cell expression of MazF-ec promotes cleavage of and thus degradation of at least a substantial portion of the host cell RNA transcripts.

Another aspect of the present invention relates to methods of producing a protein containing an amino acid analogue(s) within the primary amino acid sequence of the protein via utilization of a cell-based expression system, wherein the cell-based expression system as presented effectively prevents incorporation of the respective amino acid analogue into host cellular or ‘non-target’ proteins. As exemplified herein, the ability to restrict incorporation of the non-natural or amino acid analog at least substantially to the modified target protein promotes stable and effective host cell-based expression of the modified target protein containing the respective amino acid analogue.

An additional aspect of the present invention relates to methods of producing a protein containing an amino acid analogue(s) within the primary amino acid sequence of the protein via utilization of a cell-based expression system, wherein the cell-based expression system as utilized effectively prevents de novo biosynthesis of the particular proteinogenic amino acid targeted by substitution. One embodiment of this portion of the invention relates to effectively preventing de novo biosynthesis of the particular proteinogenic amino acid targeted by utilizing a cell-based expression system incorporating any known amino acid auxotroph or auxotrophs, which is strategically selected by the artisan from the group consisting of a glycine (Gly) auxotroph, a valine (Val) auxotroph, a leucine (Leu) auxotroph, an isoleucine (Ile) auxotroph, a proline (Pro) auxotroph, a phenylalanine (Phe) auxotroph, a tyrosine (Tyr) auxotroph, a tryptophan (Trp) auxotroph, a serine (Ser) auxotroph, a threonine (Thr) auxotroph, a cysteine (Cys) auxotroph, a methionine (Met) auxotroph, as asparagine (Asn) auxotroph, a glutamine (Gln) auxotroph, a lysine (Lys) auxotroph, an arginine (Arg) auxotroph, a histidine (His) auxotroph, an aspartate (Asp) auxotroph, a glutamate (Glu) auxotroph, an alanine (Ala) auxotroph, ornithine (Orn) auxotroph and if applicable, selenocysteine (Sec) auxotroph.

An additional aspect of the present invention Further, according to the present invention, peptides or peptide derivatives can be synthesized by previously reacting an amino acid with aminoacyl-tRNA synthetase and reacting the resulting reaction mixture with an amino acid derivative. Amino acids suitably used for previously reacting with aminoacyl-tRNA synthetase include tyrosine, alanine, leucine, isoleucine, phenylalanine, methionine, lysine, serine, valine, asparagine, aspartic acid, glycine, glutamine, glutamic acid, cysteine, threonine, tryptophane, histidine or proline, etc., which may be in the L-compound and D-compound form. Further, useful amino acid derivatives include esters, thioesters, amides and hydroxamides, etc. of various amino acids, for example, α-amino acids such as glycine, alanine, leucine, isoleucine, phenylalanine, glutamic acid, glutamine, norleucine, cysteine, tyrosine, arginine,valine, lysine, histidine, aspartic acid, asparagine, methionine, tryptophane, arginine canavanine, threonine, ornithine, or selenoscysteine etc., β-amino acids such as β-alanine or β-aminoisobutyric acid, etc., nitrogen containing γ-amino acids such as creatine, etc., γ-amino acids such as piperidic acid, etc., and ε-amino acids such as ε-aminocapronic acid, etc. However, the amino acid derivatives are not limited to the above described compounds, if they have a free amino group. Various esters can be used such as simple hydrocarbon esters including methyl, ethyl, propyl, cyclohexyl, phenyl or benzyl ester as well as esters prepared by esterifying the 3′-OH of tRNA with the above described amino acids. Further, useful amides include free amides as well as oligopeptides and polypeptides wherein amide bonds are formed with different kinds or the same kinds of amino acids. It is also possible to use esters, thioesters, hydroxamides and ethers of the above described oligopeptides and polypeptides. Further, the above described amino acid derivatives may be used in the form of an aqueous solution or in a solid state.

The present invention further relates to methods of producing a protein containing an amino acid analogue(s) within the primary amino acid sequence of the protein via utilization of a prokaryotic cell-based expression system, including but in no way limited to an Escherichia coli cell-based expression system, wherein such a cell-based expression system (i) contains a trans-acting factor which substantially inhibits or degrades host cell RNA transcripts in conjunction with an expressed mRNA encoding the target protein which is not susceptible to such degradation; (ii) substantially prevents incorporation of the respective amino acid analogue into host cellular or non-target proteins, and (iii) substantially prevents de novo biosynthesis of the particular proteinogenic amino acid targeted for replacement or substitution by an amino analogue.

The present invention further relates to methods of producing a protein containing an amino acid analogue(s) within the primary amino acid sequence of the protein via utilization of a prokaryotic cell-based expression system, including but in no way limited to an Escherichia coli cell-based expression system, wherein such a cell-based expression system (i) presenting a trans-acting factor which substantially inhibits or degrades host cell RNA transcripts but which does not impart such cleavage or inhibiting activity when utilized in conjunction with an expressed mRNA encoding the target protein; (ii) substantially prevents incorporation of the respective amino acid analogue into host cellular or non-target proteins during host cell culture, and (iii) effectively prevents de novo biosynthesis of the particular proteinogenic amino acid targeted for replacement with a respective amino acid analogue though utilization of an appropriate amino acid auxotroph to prevent such unwanted de novo biosynthesis of the amino acid targeted for this translational substitution by that respective amino acid analogue.

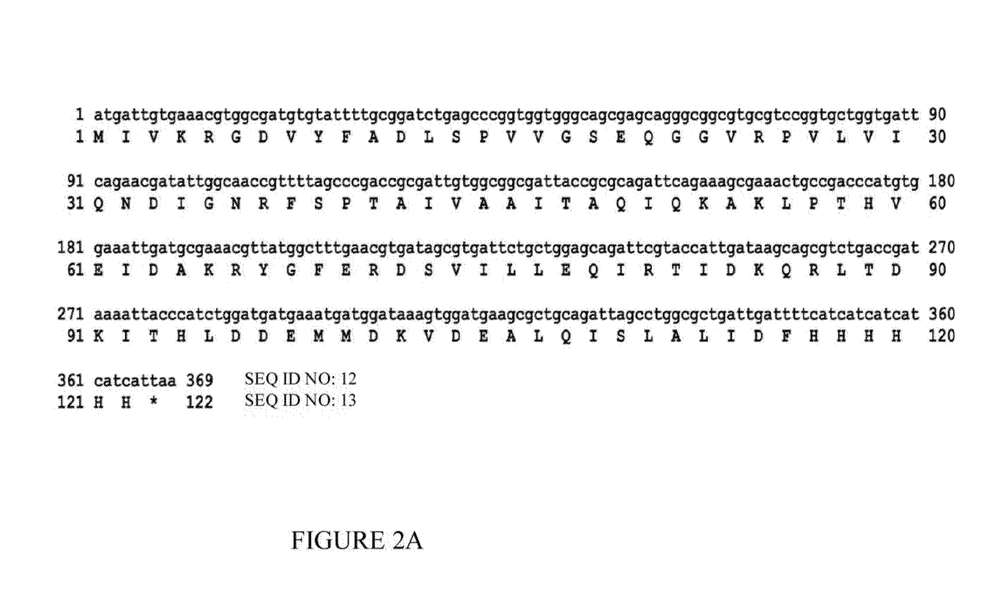

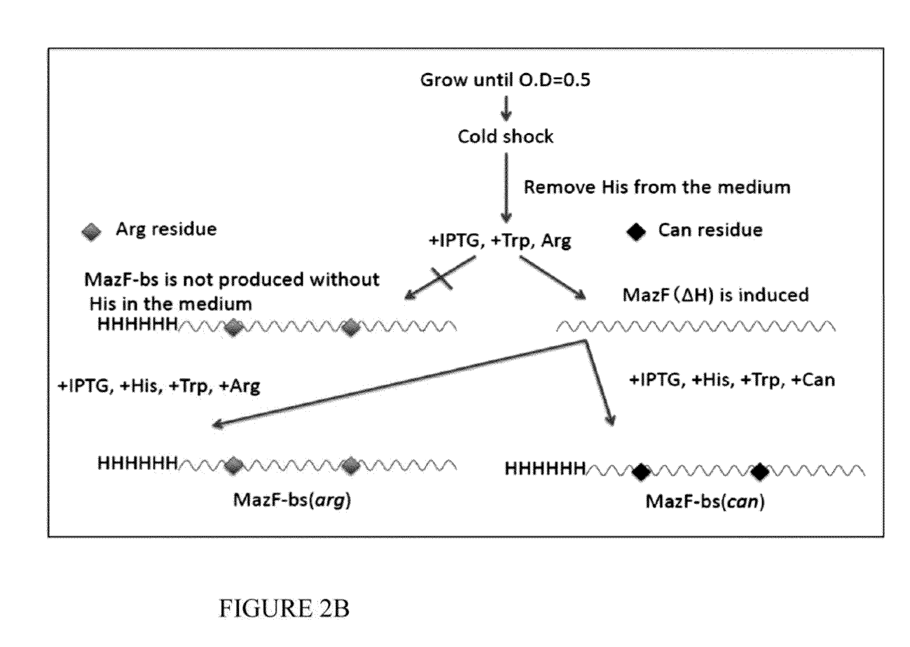

Thus, the present invention relates to methods of producing functional proteins via substitution of one or more natural amino acids with an amino acid analogue which comprises utilizing a cell-based expression system, as exemplified herein, whereby a first component of the system substantially inhibits or removes host cellular RNA transcripts so as to prevent host cell translation and subsequent generation of host proteins containing the amino acid analogue; a second component of the system effectively preventing incorporation of the respective amino acid analogue into host cellular or non-target proteins; and a third component of this cell-based expression system effectively preventing de novo biosynthesis of the particular proteinogenic amino acid targeted by substitution though utilization of an appropriate amino acid auxotroph to prevent such unwanted de novo biosynthesis of the amino acid targeted for translational substitution by a respective amino acid analogue. To this end, a particular embodiment of this portion of the present invention relates to use of an Escherichia coli cell-based expression system referred to herein as a Single Protein Production (SPP) system, as exemplified in detail herein, as shown schematically at FIG. 2B, and as further disclosed in Vaiphei et al. (2010) Appl. Environ. Microbiol. 76: 6063-6068 and Suzuki et al. (2006) J. Biol. Chem. 281: 37599-37565, whereby both references are incorporated by reference herein in their entirety.

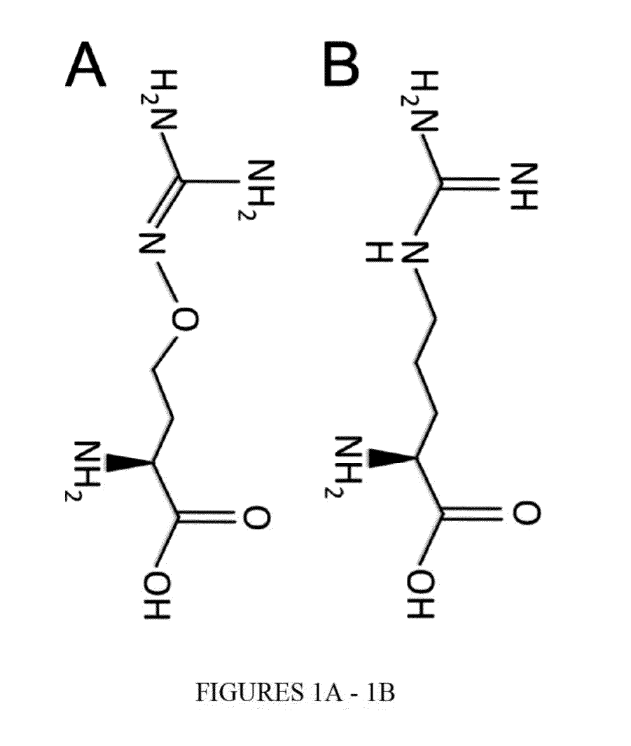

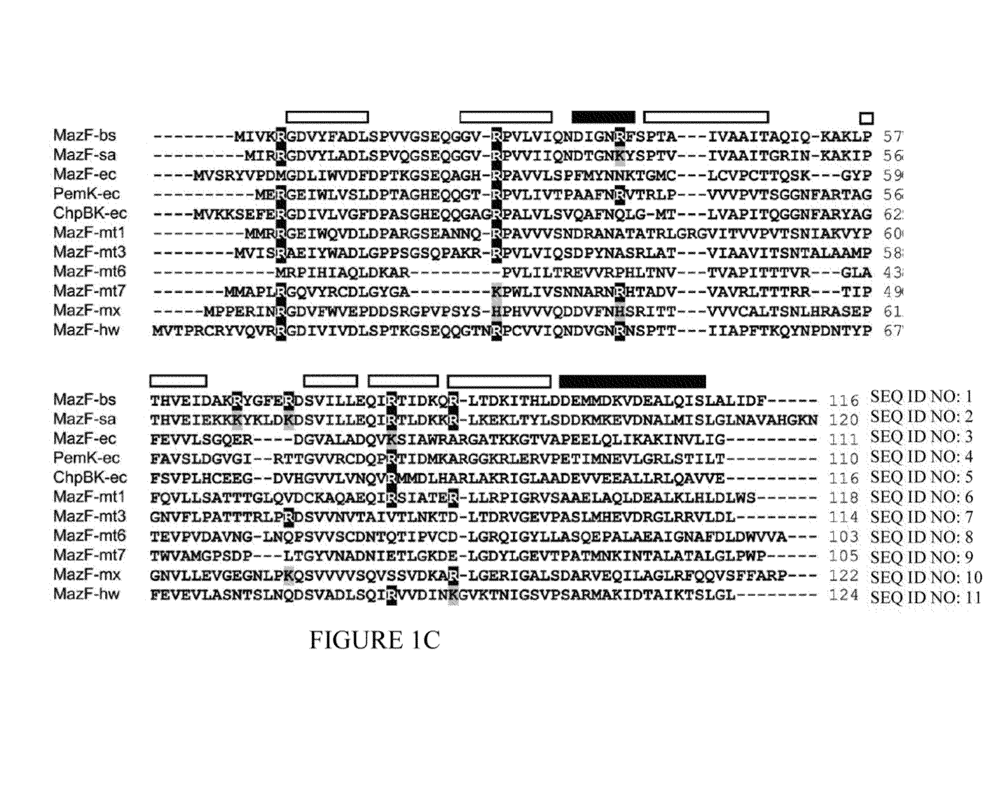

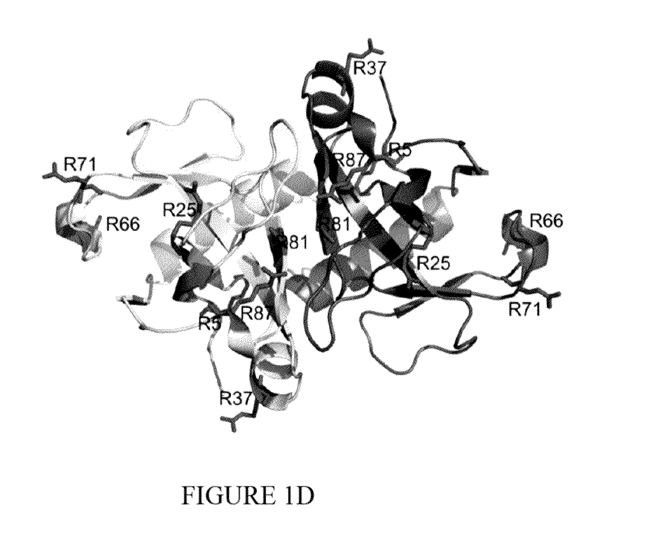

The present invention further relates to use of a prokaryotic cell-based expression system as disclosed herein whereby a target protein as disclosed in FIG. 1C is generated which substitutes or replaces each arginine (Arg) residue with canavanine (Can), including but in no way limited to an Escherichia coli cell-based expression system, wherein such a cell-based expression system (i) provides for a protein which selectively inhibits, removes and/or degrades host cell RNA transcripts (with no concomitant activity against the target protein mRNA transcripts) in order to prevent translation of host cell proteins containing amino acid analogue residues; (ii) effectively preventing incorporation of the respective amino acid analogue into host cellular or non-target proteins, and (ii) effectively preventing de novo biosynthesis of the particular proteinogenic amino acid targeted by substitution though utilization of an appropriate arginine auxotroph.

The present invention further relates to the use of a prokaryotic cell-based expression system as disclosed herein whereby a target protein selected from the group consisting of MazF-bs and MazF-hw, as disclosed in FIG. 1C, are generated which substitutes or replaces each arginine (Arg) residue with canavanine (Can) to produce MazF-bs (can) and MazF-hw (can), including but in no way limited to an Escherichia coli cell-based expression system, wherein such a cell-based expression system (i) effectively inhibits or removes host cell RNA transcripts in order to prevent translation of host cell proteins containing amino acid analogue residues; (ii) effectively prevents incorporation of the respective amino acid analogue into host cellular or non-target proteins, and (ii) effectively prevents de novo biosynthesis of the particular proteinogenic amino acid targeted by substitution though utilization of an appropriate arginine auxotroph. And a particular embodiment of this portion of the invention relates to utilization of this methodology to generate MazF-bs (can), whereby each of the seven arginine residues is replaced with a canavanine residue. It will be understood by the artisan that the exemplified replacement of arginine residues for the non-natural analogue canavanine to express MazF-bs (can) is not meant to be limiting in any fashion. Instead, this exemplification shows that the methodology as disclosed herein is now readily available for the directed replacement of one or more of the twenty proteogenic amino acids (namely, Ala, Gly, Val, Leu, Ile, Pro, Phe, Tyr, Trp, Ser, Thr, Cys, Met, Asn, Gln, Lys, Arg, His, Asp and Glu) with any art-recognized amino acid analogue, so long as any such amino acid analogue is recognized by a respective host cell aminoacyl-tRNA synthetase.

The present invention also relates to the use of a prokaryotic cell-based expression system as shown schematically in FIG. 2B and as exemplified herein, to produce MazF-bs (can), a target protein where all seven arginine residues (as disclosed in FIG. 1C and FIG. 2A) have been replaced with a canavanine residue.

The present invention further relates to isolated proteins which comprise one or more substituted or replaced amino acid(s) within the primary amino acid sequence of the isolated protein with an amino acid analogue(s) as compared to the respective amino acid sequences disclosed in FIG. 1A, namely, MazF-bs, MazF-sa, MazF-ec, PemK-ec, ChpBK-ec, MazF-mt1, MazF-mt3, MazF-mt6, MazF-mt7, MazF-mx, and MazF-hw.

The present invention further relates to isolated proteins which comprise an amino acid sequence whereby each arginine residue has been substituted with a canavanine residue compared to the respective amino acid sequences disclosed in FIG. 1A, namely, MazF-bs, MazF-sa, MazF-ec, PemK-ec, ChpBK-ec, MazF-mt1, MazF-mt3, MazF-mt6, MazF-mt7, MazF-mx, and MazF-hw, resulting in MazF-bs (can), MazF-sa (can), MazF-ec (can), PemK-ec (can), ChpBK-ec (can), MazF-mt1 (can), MazF-mt3 (can), MazF-mt6 (can), MazF-mt7 (can), MazF-mx, and MazF-hw (can), respectively. To this end, the protein sequence of each respective modified target protein is easily noted by simply referring to FIG. 1A and directly substituting each arginine residue with a canavanine residue.

A particular embodiment of this portion of the present invention relates to isolated proteins selected from the group consisting of MazF-bs (can) and MazF-hw (can), respectively; which again, the protein sequence of both MazF-bs (can) and MazF-hw (can) is determined simply by referring to FIG. 1A and directly substituting each arginine residue with a canavanine residue.

Another particular embodiment of this portion of the present invention relates to the isolated protein MazF-bs (can), which substitutes canavanine for each of the seven arginine residues of MazF-bs, as disclosed in FIG. 1A; and again, is determined simply by referring to FIG. 1A and directly substituting each arginine residue with a canavanine residue.

The present invention thus also relates to a method of using a SPP system for replacing at least one arginine with at least one canavanine in a peptide or protein, comprising:
a. transforming BL21(DE3) (ΔargHΔtrpCΔhisB) cells with a vector containing an ACA-less gene for a Protein Y together with pACYCmazF(ΔH);
b. inducing protein expression of MazF(ΔH);
c. incorporating canavanine into Protein Y;
d. purifying Protein Y(arg) and Protein Y(can); and
e. further purifying Protein Y(can).

As noted, the present invention also relates to a composition comprising MazF-bs(can).

Another embodiment of the present invention is a method of using a SPP system for replacing at least one arginine with at least one canavanine in a peptide or protein, comprising:
a. transforming BL21(DE3) (ΔargHΔtrpC≢hisB) cells with a vector containing an ACA-less gene for a protein (Protein Y) together with pACYCmazF(ΔH);
b. inducing expression of MazF(ΔH) in a medium containing Arg, Trp and His;
c. incorporating canavanine into Protein Y by replacing Arg with canavanine in the medium;
d. purifying Protein Y(can); and
e. purifying Protein Y(arg) from the medium containing Arg.

As noted, the present invention also relates to a composition comprising Protein Y(can).

In an embodiment, at least one arginine in a peptide or protein is replaced with at least one canavanine. In another embodiment, each arginine is replaced with a canavanine.

### EXAMPLES

Strain construction—E. coli BL21(DE3) (ΔargHΔtrpCΔhisB) was constructed from E. coli BL21(DE3) (ΔtrpCΔhisB) by P1 transduction using the ΔargH strain from the Keio collection.

Plasmid construction—The gene for MazF-bs with a C-terminal His-tag (FIG. 2A) was synthesized (Genescript). The gene was designed for the optimal codon usage in E. coli and to have no ACA sequences. The gene was cloned into pColdIII (SP-4).

Protein expression and purification—The BL21(DE3) (ΔargHΔtrpCΔhisB) cells were transformed with pColdIIImazF-bs together with pACYCmazF(ΔH) and grown in a 1-liter culture of M9-glucose medium in the presence of Arg (20 μg/ml), His (20 μg/ml), and Trp (20 μg/ml) at 37° C. When the A600 value reached 0.5, the culture was chilled in an ice-water bath for 5 min and incubated at 15° C. for 1 hr to acclimate the cells to cold shock conditions. Cells were harvested and washed twice with M9 medium. The cells were resuspended in 50 ml of M9-glucose medium containing Arg (20 μg/ml) and Trp (20 μg/ml) but without His. Isopropyl β-D-1-thiogalactopyranoside (IPTG; 0.5 mM) was added to induce the only expression of MazF(ΔH) followed by an additional 2 hr incubation at 15° C. Cells were harvested and washed twice with M9 medium. The cells were resuspended in 50 ml of M9-glucose medium containing His (20 μg/ml), and Trp (20 μg/ml), Can (100 μg/ml; Sigma) and IPTG (0.5 mM) to incorporate Can into MazF-bs. The cell culture was incubated at 15° C. for additional 24 hr to induce MazF-bs(can) (FIG. 2B). Cells were collected by centrifugation and subjected to SDS-PAGE followed by Coomassie Blue staining MazF-bs(arg) and MazF-bs(can) were purified from BL21(DE3) (ΔargHΔtrpCΔhisB) cells carrying pColdIIImazF-bs with use of Ni-NTA resin (Qiagen) following the manufacturer's protocol. The MazF-bs(can) and MazF-bs(arg) were further purified by ion-exchange chromatography using DEAE Sepharose (GE Healthcare).

Circular dichroism (CD) analysis—CD analysis was carried out using an AVIV Model 62DS spectropolarimeter (Aviv Associates, Inc., Lakewood, N.J.) Spectra were recorded in 2.0-nm steps between 260 and 200 nm at 4° C. with an integration time of 4 s at each wavelength, and the base line was corrected against buffer alone. Protein melting was examined at 208 nm with increasing temperature, from 0 to 90° C., in 0.3° C. steps. Protein solutions were equilibrated at each temperature point for 1.5 min, and the temperature was increased with an average rate of 0.1° C./min. The path length of the cell used was 0.1 cm and all measurements were carried out in 10 mM Tris-HCl (pH 7.8).

Cleavage of MS2 phage RNA by MazF-bs(can)—MS2 phage RNA (70 nM; Roche) was incubated with 0.5 μM of MazF-bs(arg) or 0.5 μM of MazF-bs(can) in a reaction mixture (10 μl) containing 10 mM Tris-HCl (pH 7.8), 1 mM dithiothreitol and the Protector RNase inhibitor at 37° C. for 0, 1, 5, 10, and 30 min. After denaturation in urea, the products were analyzed by electrophoresis on a 1.2% agarose gel.

Primer extension analysis—Primer extension analysis of the cleavage sites was carried out as previously described. Briefly, 0.7 μM MS2 phage RNA was incubated with 0.5 μM of MazF-bs(arg) or MazF-bs(can) in the presence of CspA protein, an RNA chaperone (20 μM) at 37° C. for 10 min in a reaction mixture (10 μl) in 10 mM Tris-HCl (pH 7.8), containing 0.2 μl of the Protector RNase inhibitor (Roche). Primer extension was carried out at 47° C. for 1 hr. The reactions were stopped by 2× stop solution (90% formamide, 50 mM EDTA, 0.05% bromophenol blue, and 0.05% xylene cyanol FF). The samples were incubated at 90° C. for 5 min prior to electrophoresis on a 6% polyacrylamide gel containing 8 M urea.

Cleavage of synthetic RNA by MazF-bs(arg) and MazF-bs(can)—Four 13-base ribonucleotides (CUCXUACAUAUCA) were synthesized, where the 4th base (X) was A, U, G or C. Additional three 13-base RNA ribonucleotides (CUCUUACAUYUCA) were synthesized, where the Y position was replaced with U, G or C. These ribonucleotides were used as substrates. The labeled substrates (0.2 μM) with [γ-32P]-ATP using T4 kinase (New England Biolabs) were incubated with 0.1 μM of MazF-bs(arg) or MazF-bs(can) for 10 min at 37° C. in a reaction mixture (10 μl) in 10 mM Tris-HCl (pH 7.8) containing 0.2 μl of the Protector RNase inhibitor. The reactions were stopped by the use of 2× stop solution. To analyze the cleavage of the synthetic RNAs, the products were analyzed by electrophoresis on a 20% polyacrylamide gel containing 8 M urea with a molecular-weight ladder.

Kinetics analysis—A 13-base ribonucleotide (CUCAUACAUAUCA) was used as a substrate. The substrate in various concentration (0.5, 1.0, 1.5, 2.0, 2.5, 3.0, 3.5, and 4.0 μM) was incubated with 1 nM of MazF-bs(arg) or 5 nM of MazF-bs(can) in a reaction mixture (10 μl) in 10 mM Tris-HCl (pH 7.8) containing 0.2 μl of the Protector RNase inhibitor. The reaction with MazF-bs(arg) was incubated for 5, 10, 15 and 20 min. The reaction with MazF-bs(can) was incubated for 30, 60, 90 and 120 min. The reaction was stopped by the use of 2× stop solution and the sample mixtures were incubated at 90° C. for 5 min prior to electrophoresis on a 20% polyacrylamide gel containing 8 M urea. The cleavage products were analyzed by software imageJ.

Competitive analysis—A 13-base ribonucleotides (CUCUUACAUAUCA) was used as substrate, and three other 13-base ribonucleotides (CUCUUACAUUUCA, CUCUUACAUCUCA, and CUCUUACAUGUCA) in which only the tenth bases are different from the substrate (shown in bold) were used to examine if these ribonucleotides are able to inhibit the cleavage of the substrate. The concentration of the substrate analogues was fixed at 1 μM, while the substrate concentrations were used at 1.0 and 4.0 μM. The substrate with and without the substrate analogues in a 10-μl reaction mixture containing 10 mM Tris-HCl (pH7.8) and 0.2 μl of Protector RNase inhibitor was incubated with 5 nM of MazF-bs(can) at 37° C. for 30, 60, 90 and 120 min. The reaction was stopped by the use of 2× stop solution and the reaction mixtures were incubated at 90° C. for 5 min prior to electrophoresis on a 20% polyacrylamide gel containing 8 M urea. The cleavage products were analyzed by imageJ.

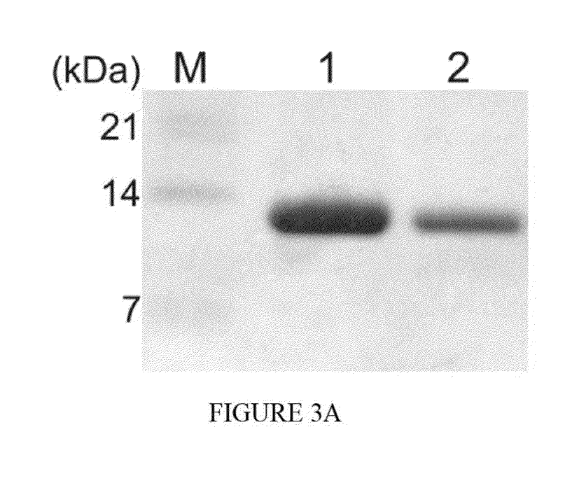

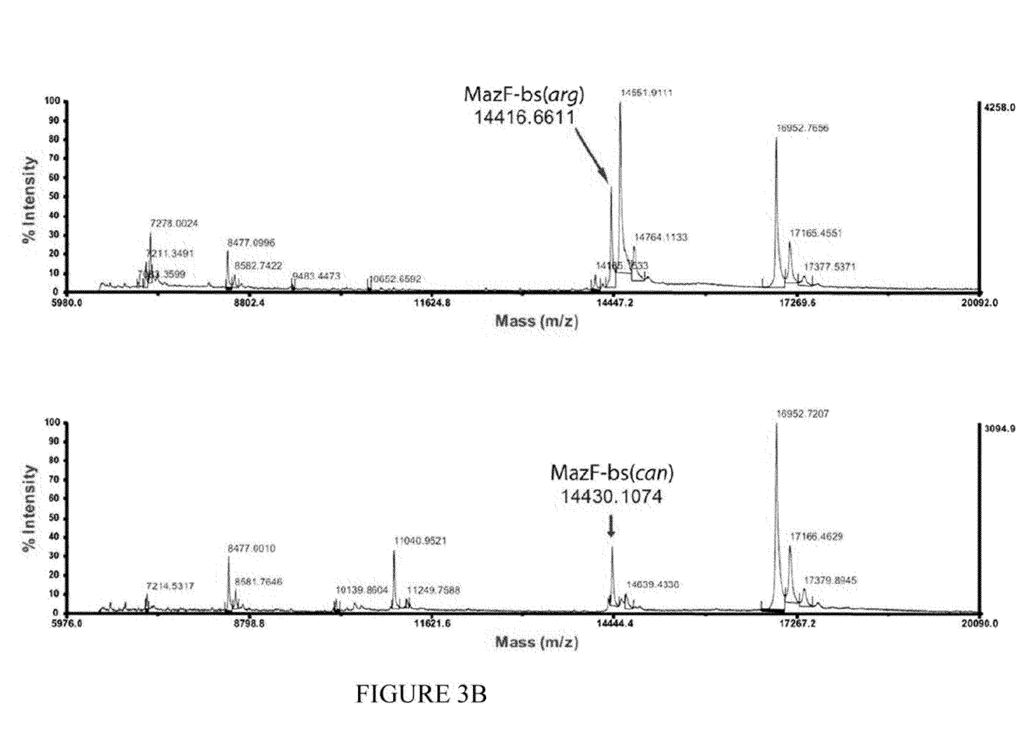

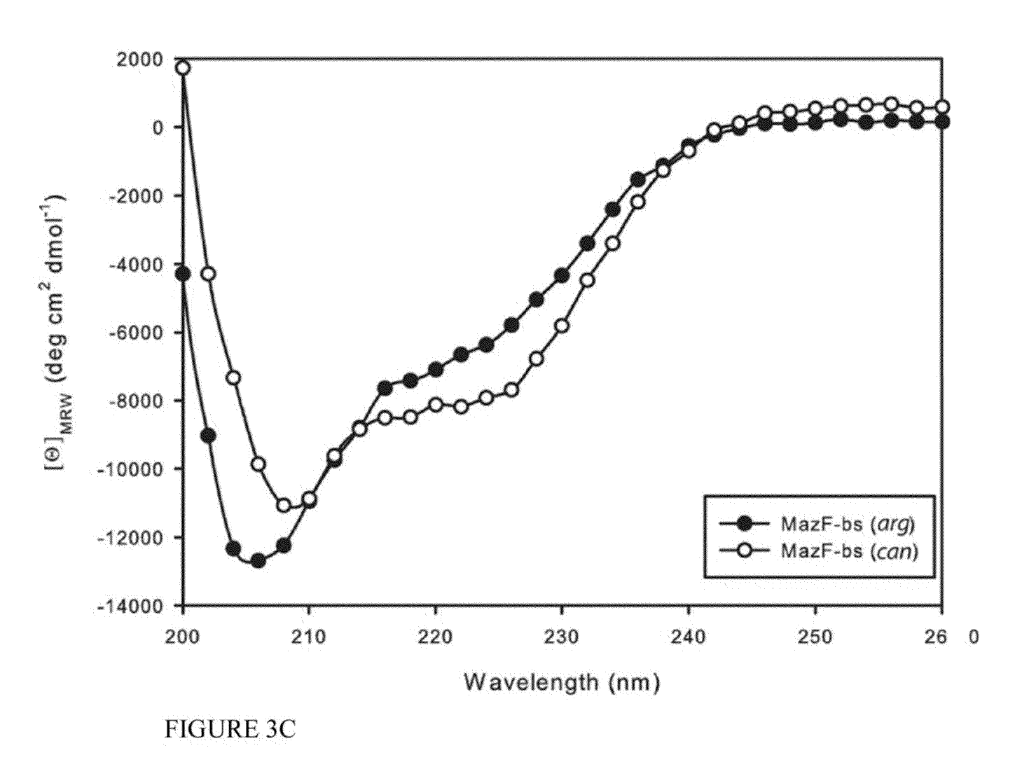

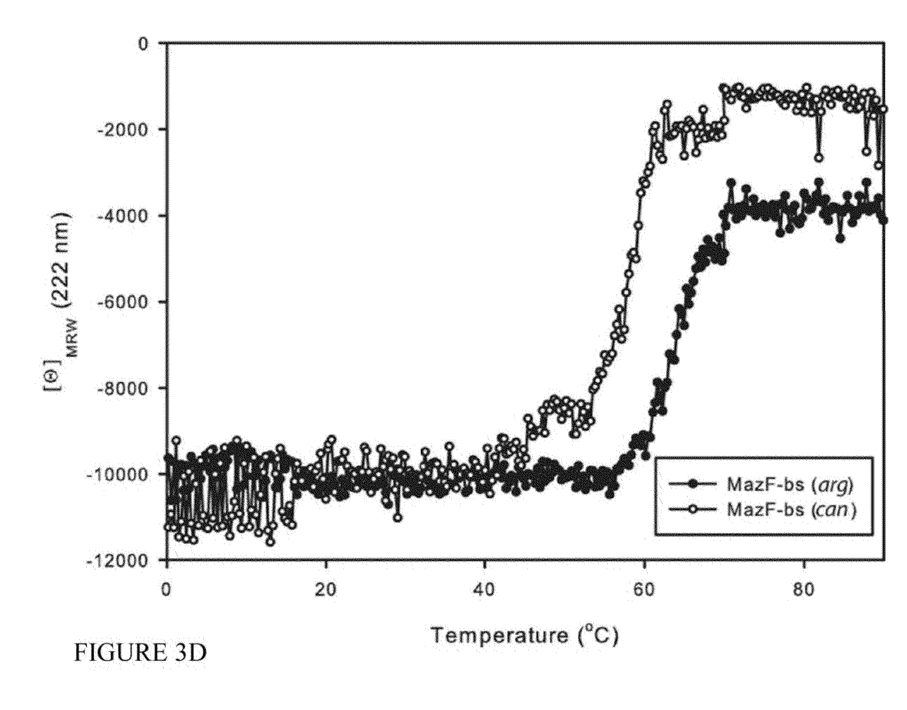

Production of MazF-bs(can) by the Single Protein Production (SPP) System—In order to replace all seven Arg residues in; MazF-bs with Can, we applied the Single-Protein Production (SPP) system with use of an Arg auxotroph. In the SPP system, E. coli cells are converted into a bioreactor producing only a target protein, in which an ACA-specific mRNA interferase, MazF-ec, from E. coli is induced to eliminate all cellular mRNAs but the ACA-less mRNA for the target protein. Therefore, the use of the SPP system enables us to avoid the cytotoxicity of Can to replace all Arg residues in MazF-bs with Can. Thus, all ACA sequences in the MazF-bs mRNA are changed to other sequences without altering the MazF-bs amino acid sequence. For the complete replacement of all Arg residues with Can, it is also important to block the biosynthesis of Arg in the cells using an Arg auxotroph. For this, the argH deletion mutation from the Keio collection was transduced into E. coli BL21(DE3) ΔtrpC, ΔhisB. The gene for MazF-bs designed to be ACA-less and codon-optimized for E. coli (FIG. 2A) was synthesized and cloned into pColdIII(SP-4) vector, yielding pColdIIImazF-bs. E. coli BL21(DE3)ΔargHΔtrpCΔhisB cells were co-transformed with pColdIIImazF-bs and pACYCmazF(ΔH). In MazF(ΔH), His-28 and Gly-27 in MazF-ec were replaced with Arg and Lys, respectively, which has no effect on the MazF-ec mRNA interferase activity. Since MazF(ΔH) thus obtained does not contain His residues, this protein can still be synthesized in E. coli BL21(DE3)ΔargHΔtrpCΔhisB cells in the absence of His in the medium containing Trp, Arg and IPTG as previously reported. Under this condition, cell growth is completely arrested and MazF(ΔH) eliminates all ACA-containing cellular mRNAs. Note that in contrast to MazF-ec produced from pACYCmazF(ΔH), MazF-bs used in the present study contains a C-terminal extension containing six His residues so that in the absence of His in the medium, the production of MazF-bs is completely blocked. Using this condition, Arg (20 μg/ml) was replaced with Can (100 μg/ml) in the medium in the presence of Trp and His (FIG. 2B). Complete replacement of Arg residues in MazF-bs with Can—After 24-hr incubation using the SPP system in the presence of Can, a new band was induced at 14 kDa, and purified protein was shown in FIG. 3A. This protein termed [MazF-bs(can)] was subsequently purified by Ni-NTA affinity chromatography and DEAE ion-exchange column chromatography (FIG. 3A). If all seven Arg residues were replaced with Can, the molecular mass of MazF-bs(can) should be larger by 13.8 Da (1.97 Da×7) than that of MazF-bs(arg). The mass spectrometry analysis revealed that MazF-bs(can) was larger by 13.4 Da than MazF-bs(arg) (FIG. 3B), indicating that 97% of Arg residues were replaced with Can or that in four out of five MazF-bs molecules the complete replacement was achieved. In the remaining one molecule, all but one Arg residue out of seven were replaced with Can.

Structural analysis of MazF-bs(can) by circular dichroism (CD) spectroscopy—The secondary structures of purified MazF-bs(arg) and MazF-bs(can) were analyzed by CD spectroscopy. MazF-bs(arg) showed minimum peaks around at 208 and 222 nm, which are characteristic for α-helical structures (23). MazF-bs(can) also showed a minimum peak at 208 nm which is higher than that for MazF-bs(arg), while the signal at 222 nm for MazF-bs(can) was lower than that for MazF-bs(arg) (FIG. 3C), indicating that α-helix contents of MazF-bs(can) slightly increased from 27.5 to 29.7%, while its β-sheet content decreased from 39.5 to 37.2%. Next, the thermal stability was examined for both proteins between 4 and 90° C. by measuring the change in ellipticity at 222 nm in the CD spectra. Notably, Tm for MazF-bs(can) was lower by approximately 4° C. than that for MazF-bs(arg) (FIG. 3D). MazF-bs(can) is likely folded in a very similar manner as MazF-bs(arg), however the substitution of Arg with Can appears to affect the α-helical structures. The hydrogen bonds between Arg-5 and Ala-112, and a salt bridge between Arg-87 and Glu-20 have been shown to stabilize the dimer formation. Although both Arg and Can contain a guanidine group, the replacement of the methylene group in Arg with oxygen in Can results in the reduction of the pKa value from 12.48 to 7.01. Therefore, the salt bridge in MazF-bs(arg) is likely to weaken substantially when all the Arg residues are replaced with Can, resulting in a less thermo-stable protein. Note that the pI value of MazF-bs changed from 6.34 to 5.86 as a result of the Arg-to-Can replacement.

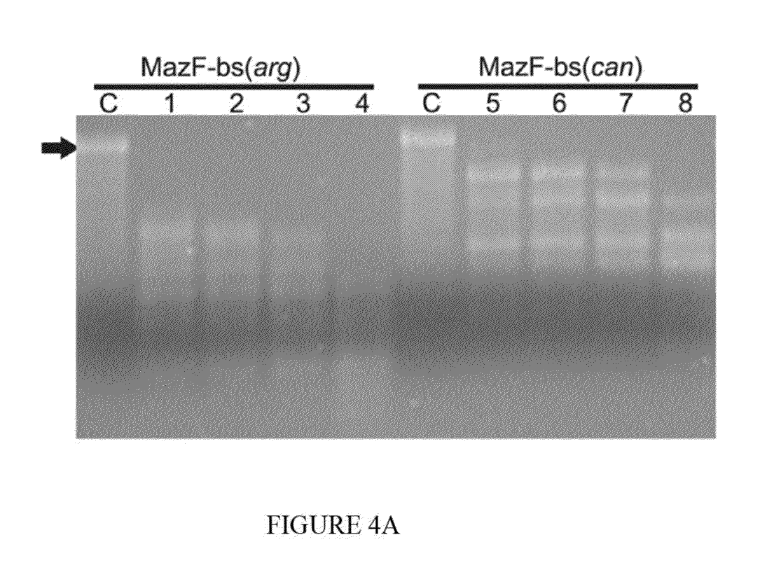

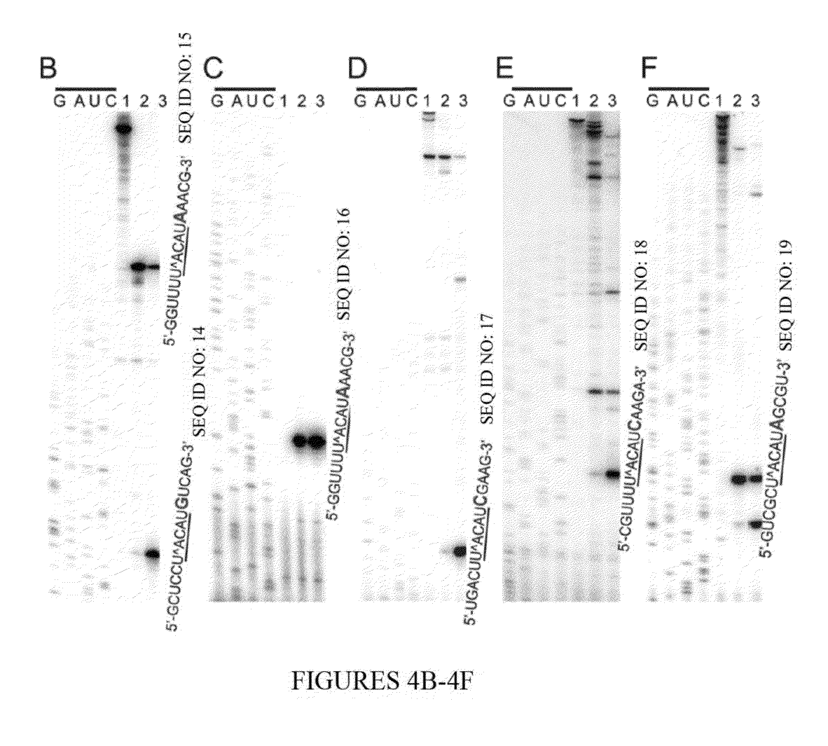

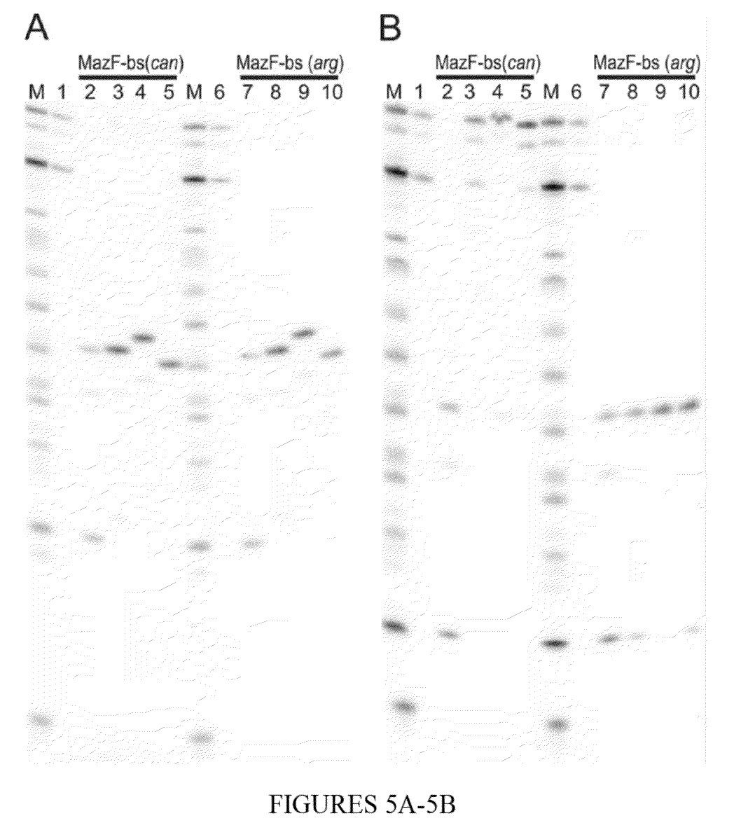

Specificity alteration of a five-base to a six-base recognition for RNA cleavage—Next, we analyzed the endoribonuclease activity of MazF-bs(can) using 3.5-kb MS2 phage RNA as a substrate. Since the RNA cleavage patterns were found to be quite different between the two enzymes (FIG. 4A), in vitro primer extension experiments were carried out to determine the exact cleavage site sequences. As shown in FIG. 4A, after incubation of the RNA with MazF-bs(can) and MazF-bs(arg) at 37° C. for 10 min, MazF-bs(arg) cleaved MS2 phage RNA at all ÛACAU sites as expected (̂ indicates the cleavage site), while MazF-bs(can) appears to cleave the MS2 RNA at ÛACAU sites, only when these sites contain one extra A residue at the 3’ end (FIG. 4B-F), indicating that MazF-bs(can) acquired a higher RNA-cleavage specificity from a five-base to a six-base cutter. To further confirm this notion, we synthesized 13-base RNA substrates covering all the possible 7-base sequences having an extra base at both sides of UACAU and confirmed that MazF-bs(can) is specific for ÛACAUA (FIG. 5A, B). Lanes 2 [MazF-bs(can)] and 7[MazF-bs(arg)] in FIG. 5A show an extra band corresponding to the product cleaved after the first C residue in addition to the cleavage product after the fifth U residue ĈUCUÛACAUAUCA (̂ indicates the cleavage sites), while no cleavage products are observed with three other ribonucleotides (CUCAUACAUAUCA, CUCGUACAUAUCA, CUCCUACAUAUCA) for both MazF-bs(can) and MazF-bs(arg) (bases which are replaced are shown in bold). Furthermore, lane 2 in FIG. 5B using MazF-bs(can) with CUCUUACAUAUCA shows an extra cleavage product (cleaved after the first C residue) in addition to the product after the fifth U residue. Lanes 7, 8 and 10 in FIG. 5B using MazF-bs(arg) also show an extra cleavage product corresponding to ĈUCUUACAUAUCA, ĈUCUUACAUGUCA, ĈUCUUACAUCUCA. These cleavages were not observed with MazF-bs(can). It is unknown at present why these substrates were cleaved by MazF-bs(arg) after the first C residue.

Kinetic study—Using UACAUA as a substrate, the Km value and the Kcat/Km value of MazF-bs(arg) were determined to be 2.0±0.2 μM and 1.0±0.2×10-2, respectively. Although the Kcat/Km value of MazF-bs(can) is approximately 5% of that of MazF-bs(arg), the Km value for MazF-bs(can) was almost identical to that of MazF-bs(arg) (see Table 1).

TABLE 1
Kinetic constants for MazF-bs(arg) and MazF-bs(can)

| | Vmax | Km | Kcat | Kcat/Km |
|---|---|---|---|---|
| | (μM/min) | (μM) | (min⁻¹) | (μM⁻¹ · min⁻¹) |
| MazF-bs(arg) | 4.2 ± 1.1 × | 2.0 ± 0.2 | 2.2 ± 0.4 × | 1.0 ± 0.2 × |
| | 10⁻² | | 10⁻² | 10⁻² |
| MazF-bs(can) | 8.4 ± 1.2 × | 1.8 ± 0.3 | 8.4 ± 1.2 × | 5.0 ± 1.0 × |
| | 10⁻³ | | 10⁻⁴ | 10⁻⁴ |

The difference in the Kcat/Km values is likely due to the charging status of the guanidine groups of Can residues in MazF-bs(can). Since MazF-bs(can) became six-base specific, cleaving at ÛACAUA, but not UACAUG, UACAUC and UACAUU (FIG. 5A, B), we next examined if the cleavage of ÛACAUA is inhibited by substrate analogues having different bases at the sixth position (UACAUU, UACAUC and UACAUG), which are not cleavable by MazF-bs(can), and found that there was no inhibition of the cleavage reaction by UACAUG, UACAUC, and UACAUU, indicating the A residue at the sixth position plays a critical role for the substrate binding to the enzyme (Table 2). It was found that there was no inhibition of the cleavage reaction by UACAUG, UACAUC, and UACAUU, even if the inhibitor-to-substrate ratio increased (Table 2).

TABLE 2
Relative cleavage activity of MazF-bs(can) using CUCUUACAUAUCA as a
substrate in the presence and the absence of substrate analogues having
different bases at the sixth position (CUCUUACAUCUCA, CUCUUACAUGUCA and
CUCUUACAUUUCA)

| | CUCUUACAUAUCA | | | |
|---|---|---|---|---|
| | only | +CUCUUACAUCUCA | +CUCUUACAUGUCA | +CUCUUACAUUUCA |
| CUCUUACAUAUCA: | 1.0 | 1.1 | 1.0 | 1.1 |
| substrate analogue = 1:1 | | | | |
| CUCUUACAUAUCA: | 1.0 | 1.1 | 1.1 | 1.2 |
| substrate analogue = 4:1 | | | | |

Amino acid analogues are toxic for cells, because they are incorporated into cellular proteins producing structurally and functionally abnormal proteins, which results in cell growth arrest and eventual cell death. Therefore, the simple addition of an amino acid analogue into a culture medium does not yield a protein in which all the residues of a specific amino acid in the protein are replaced with its analogue.

Furthermore, to achieve the complete replacement of all the residues of a particular amino acid in a protein, it is important to completely suppress the biosynthesis of that particular amino acid. Therefore, in order to achieve the complete replacement of a specific amino acid in a protein, there are two essential requirements; first, the incorporation of an amino acid analogue into any other cellular proteins but the target protein has to be completely prevented. Secondly, the de novo biosynthesis of that particular amino acid should be completely inhibited. To achieve the replacement of all seven Arg residues in MazF-bs with Can, we used the SPP system for the first requirement so that Can incorporation into cellular proteins but MazF-bs was prevented, while maintaining the biosynthetic function of the cells. The second requirement was achieved by using an Arg auxotroph. The use of SPP system for the replacement of all Arg residues in a protein with Can seems to be crucial as Can incorporation into other cellular proteins likely affects their functions leading to severe inhibitory effects on various biosynthetic reactions including protein synthesis. The present system, therefore, can be used for other toxic amino acid analogues as far as they can be recognized by E. coli aminoacyl-tRNA synthetases. The second requirement for the present system is the use of an amino acid auxotroph to avoid the incorporation of a natural amino acid into a target protein. The use of the SPP system in combination with amino acid auxotroph strains thus opens a new avenue to create proteins of unprecedented novel structures and functions without genetic manipulation of tRNAs and aminoacyl tRNA synthetases.

MazF-bs(can) was not able to cleave RNA at the original MazF-bs(arg) five-base sequence, UACAU, thus requiring one extra A residue at the 3′ end. The Km value of MazF-bs(can) using UACAUA as a substrate is almost identical to that of MazF-bs(arg) using UACAU as a substrate, suggesting that the substrate binding affinity in MazF-bs(can) was compensated by an extra A residue at the 3′ end of the substrate. Notably, however, the cleavage activity of MazF-bs(can) was reduced to approximately 5% of MazF-bs(arg) (Table 1). The MazF-bs functions as a dimer, and its interphase is predicted to be involved with RNA binding and catalysis. Since out of total seven Arg residues in MazF-bs, R25, R81 and R87 are located in the interphase between the two monomers in a dimer (FIG. 1D), some or all these three residues may have critical roles in the specific RNA sequence recognition and the enzymatic activity. At present it is not known if the other four Arg residues also play roles in MazF-bs function.

Unless otherwise defined, all technical and scientific terms used herein have the same meaning as commonly understood by one of ordinary skill in the art to which this invention belongs. Although methods and materials similar or equivalent to those described herein can be used in the methods and materials are described herein. All publications, patent applications, patents and other references mentioned herein are incorporated by reference in their entirety. In the case of conflict, the present specification, including definitions, will control. In addition, the materials, methods and examples are illustrative only and are not intended to be limiting.

Other features and advantages of the invention will be apparent from the detailed description, and from the claims.

What is claimed is:

1. A method of producing a target protein containing the targeted replacement of a natural amino acid with an amino acid analogue within a cell-based expression system, wherein the method comprises
a) presenting a trans-acting factor which substantially inhibits or degrades host cell RNA transcripts in conjunction with an expressed mRNA encoding the target protein which is not susceptible to such degradation;
b) preventing substantial incorporation of the respective amino acid analogue into host cellular or non-target proteins;
c) preventing substantial de novo biosynthesis of a specific proteinogenic amino acid which is targeted for substitution with an amino acid analogue.

2. The method of claim 1 wherein the cell-based expression system is a prokaryotic expression system.

3. The method of claim 2 wherein the cell-based prokaryotic expression system is an Escherichia coli cell-based expression system.

4. The method of claim 3 wherein de novo biosynthesis of the replaced natural amino acid is inhibited within the host cell by using a host cell which is auxotrophic for the normal amino acid.

5. The method of claim 1 wherein the expression vector of step a) expresses MazF-ec, which substantially removes host cell mRNA transcripts.

6. The method of claim 5, wherein the mRNA encoding the target protein is resistant to degradation by MazF-ec.

7. The method of claim 1 wherein de novo biosynthesis of the particular proteinogenic amino acid targeted for replacement or substitution is substantially prevented though utilization of an appropriate amino acid auxotroph.

8. A method of producing a target protein containing the targeted replacement of a natural amino acid with an amino acid analogue within a cell-based expression system, which comprises:
a) introducing into a host cell an expression vector which expresses a protein which substantially cleaves host mRNA;
b) introducing into the host cell an expression vector encoding a mRNA transcript which expresses the target protein of interest, the mRNA encoding the target protein being resistant to cleavage by the protein of step a);
c) inducing expression of the protein of step a) within the host cell;
d) removing from the host cell culture environment the natural amino acid targeted for replacement;
e) adding to the host cell culture environment the amino acid analogue;
f) inducing expression of the target protein of step b) within the host cell; and,
g) purifying the expressed target protein of step f) away from the host cell, wherein (i) incorporation of the amino acid analog(s) into host cellular proteins other than the target protein is prevented within the host cell, and (ii) de novo biosynthesis of the replaced natural amino acid is inhibited within the host cell.

9. The method of claim 8 wherein the cell-based expression system is a prokaryotic expression system.

10. The method of claim 9 wherein the cell-based prokaryotic expression system is an Escherichia coli cell-based expression system.

11. The method of claim 10 wherein de novo biosynthesis of the replaced natural amino acid is inhibited within the host cell by using a host cell which is auxotrophic for the normal amino acid.

12. The method of claim 8 wherein the expression vector of step a) expresses MazF-ec, which substantially removes host cell mRNA transcripts.

13. The method of claim 12, wherein the mRNA encoding the target protein is resistant to degradation by MazF-ec.

14. The method of claim 8 wherein de novo biosynthesis of the particular proteinogenic amino acid targeted for replacement or substitution is substantially prevented though utilization of an appropriate amino acid auxotroph.

15. An isolated protein selected from the group consisting of MazF-bs (can), MazF-sa (can), MazF-ec (can), PemK-ec (can), ChpBK-ec (can), MazF-mt1 (can), MazF-mt3 (can), MazF-mt6 (can), MazF-mt7 (can), MazF-mx, and MazF-hw (can), represented by FIG. 1A whereby each arginine residue is substituted with a canavanine residue.

16. An isolated protein of claim 15 selected from the group consisting of MazF-bs (can) and MazF-hw (can).

17. The isolated protein of claim 16 which is MazF-bs (can).

18. A method of using a SPP system for replacing at least one arginine with at least one canavanine in a peptide or protein, comprising:
transforming BL21(DE3) (ΔargHΔtrpCΔhisB) cells with a vector containing an ACA-less gene for a protein (Protein Y) together with pACYCmazF(ΔH);
inducing expression of MazF(ΔH) in a medium containing Arg, Trp and His;
incorporating canavanine into Protein Y by replacing Arg with canavanine in the medium;
purifying Protein Y(can); and
purifying Protein Y(arg) from the medium containing Arg.

19. The method of claim 19, wherein each arginine is replaced with a canavanine.

20. A composition comprising Protein Y(can) and Protein Y(arg).
